(19) United States
(12) Patent Application Publication
(10) Pub. No.: US 2009/0053312 A1
(43) Pub. Date: Feb. 26, 2009

(54) Methodology and Composition for a Skin Lubricant

(75) Inventor: Dieter R. Berndt, Reno, NV (US)

Correspondence Address: LONG & CHYBIK, 1575 DELUCCHI LANE, SUITE 32, RENO, NV, 89502, US

(73) Assignee: HIGH DESERT PHARMACEUTICALS, INC., Reno, NV (US)

(21) Appl. No.: 11/969,191

(22) Filed: Jan. 3, 2008

Related U.S. Application Data
(60) Provisional application No. 60/965,350, filed on Aug. 20, 2007.

(51) Int. Cl. A61K 9/10; A61K 31/045; A61Q 19/00

(52) U.S. Cl. 424/484; 514/724

(57) ABSTRACT

One possible embodiment of the invention could be a method of lubricating human skin comprising of the following steps, but not necessarily in the order shown: providing a composition of a gel formed from cross-linking siloxane polyethers, the gel being further micronized in one or more low viscosity, silicone fluids; applying an effective amount of the composition to a portion of the exterior surface of a skin; and moving the portion within at least a part of an elastic and flexible skin contact article.

### CROSS-REFERENCES TO RELATED APPLICATIONS

This application claims the benefit under 35 U.S.C. § 119(e) of U.S. Provisional Patent Application No. 60/965,350, filed on Aug. 20, 2007, contents of which are relied upon and incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable.

### FIELD OF THE INVENTION

The present invention may relate to a methodology and composition for lubricating human skin, namely the lubrication and subsequent insertion of a portion of the human body so as to partially encapsulating the portion within into a flexible and elastic skin contact article.

### BACKGROUND

The skin may be considered the largest organ of the human body, providing a protective cover from injury and microscopic infection; a means of cooling; and other useful attributes. The skin could excrete moisture in the form of sweat as a part of the body's cooling capability as well as secret oils as part of external skin maintenance. The accumulated moisture and oils can become a source of irritation, adhesion, and friction as it relates to elastic and flexible skin contact articles, namely those flexible and elastic items that may be used to generally partially encapsulate a portion of the human body when article(s) generally comes into contact with the skin body. Examples of these articles can be found in the medical and healthcare fields in the form of safety aids, such as examination gloves (e.g., latex or nitrile gloves) as used by healthcare professionals. Further healthcare examples could be in the form of certain prosthetic devices used to treat medical conditions and the like, such as compression hoses for diabetic patients. In industry, these articles could be various types of glove generally used for object handling and manipulation in relation to concerns for industrial hygiene (e.g., food preparation) or worker safety (e.g., contamination prevention for the worker or workplace.)

The presence of accumulated sweat and oils on the skin surface may heighten surface friction or adhesion of the skin and generally interfere with the initial fitting and subsequent re-donning of the skin contact articles. For example, when the skin of the hand becomes moist or sweaty, the elastic and flexible skin contact articles, such as thin latex gloves and the like may frequently rip apart when the hand is being placed in the glove due to the resulting increased skin friction and adhesion. Further, an application of compression wear (e.g., compression hose) by a diabetic or heart trouble patient to one or more of their limbs, in light of the article's tight fitting nature, may become a very exhaustive chore for the patient who may already be in a weakened physical condition.

Earlier attempts to apply lubricants to the areas of the skin's surface to reduce the skin's friction and adhesion qualities in relation to the application of flexible and elastic skin contact articles appears to have an inherent limitation of increasing, rather than decreasing, these undesired skin surface characteristics. For example, some hydrophilic compounds (e.g. generally aqueous-based compounds) when used as skin lubricants and are applied to a portion of the external surface of the skin, may become come tacky or sticky as water evaporates from the applied compound. Some hydrophobic compounds (e.g. generally oil-based compounds) when used as skin lubricants may be messy to apply and further impart a greasy and uncomfortable feeling to the user when applied to the skin. Additional products that may be adapted for use as skin lubricants may be a combination of the hydrophobic and hydrophilic compounds such as skin lotions and creams, which generally do little in providing the necessary skin lubrication suitable for the application of elastic and flexible skin contact devices to the human body.

What is needed therefore is a skin lubricant compound and methodology of use wherein the compound could provide effective skin lubrication for the application and re-donning of elastic and flexible skin contact articles that could be used to at least partially encapsulate one or more portions of the human body. Such a skin lubricant compound could be comprised of low molecular weight, polymeric silicone gels to provide, when applied, an initial silky feeling to the user while maintaining a high, but adjustable viscosity. These polymeric silicone gels could be further prepared in volatile silicone to hold them in solution for controlled spreadability and a high viscosity and a smooth silky feel on dry-down.

### SUMMARY OF ONE EMBODIMENT OF THE INVENTION

### Advantages of One or More Embodiments of the Present Invention

The various embodiments of the present invention may, but do not necessarily, achieve one or more of the following advantages:

to provide a skin lubricant that may be comprised of a low molecular weight polymeric silicone gel that is sheared and then mixed into one or more silicone fluids;

to provide a skin lubricant that may be comprised of a mono-oxyalkylene-functional silicone having at least one carbinol functional group;

the ability to lubricate the skin which generally gives the user a dry, silky, non-greasy feel when initially applied to the skin;

to provide a lubricant that will not generally chemically react to the skin or a flexible and elastic skin contact article that may be placed in contact with the lubricated skin;

to provide a skin lubricant that is generally surfactant free;

to provide a skin lubricant that is substantially hydrophobic in nature to resist entry of the lubricant into the blood stream of the user or applicant through wounds or other breaks in skin surface;

to provide a skin lubricant that generally utilizes a mono-oxyalkylene-functional silicone having at least one carbinol functional group; and

the ability to substantially apply a composition of a siloxane gel micronized in one or more silicone fluids to a skin surface for the generally easier application of elastic and flexible skin contact articles.

These and other advantages may be realized by reference to the remaining portions of the specification, claims, and abstract.

### BRIEF DESCRIPTION OF ONE EMBODIMENT OF THE PRESENT INVENTION

One possible embodiment of the present invention provides for a method of lubricating the human skin comprising of the following steps, but not necessarily in the order shown: providing a composition of a gel formed from cross-linked siloxanes micronized in one or more silicone fluids; applying an effective amount of the composition to a portion of the exterior surface of a skin; and reducing the frictional qualities of the portion.

Another embodiment of the present invention could be a method for using a skin lubricating composition comprising of the following steps, but not necessarily in the order shown: providing a skin lubrication composition comprising at least one silicone gel selected from a group of silicone gels consisting of: (i) a gel formed from a silicone and a hydrosilylation compatible solvent wherein said silicone is prepared by the hydrosilylation of a linear alkenyl polyorganosiloxane and a hydride resin; (ii) a gel formed as a reaction product of an epoxy functional hydrido-siloxane, said reaction product being formed in an epoxy-gel formation compatible solvent; (iii) a gel formed from a silicone and a hydrosilylation compatible solvent, wherein said silicone is prepared by the hydrosilylation of a linear hydrogen organopolysiloxane having two or more hydride functionalities per molecule and an .alpha., .omega. reactive organic molecule possessing two or more reactive functionalities per molecule; and (iv) a gel formed as a reaction product of a vinyl functional hydrido-siloxane in a hydrosilylation compatible solvent; applying an effective amount of the composition to a portion of the exterior surface of a skin; and contacting the portion with an elastic skin contact article.

The above-description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description or as illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1 is substantially showing a flow chart of one possible method for using the present invention.

### DESCRIPTION OF CERTAIN EMBODIMENTS OF THE PRESENT INVENTION

In the following detailed description of the preferred embodiments, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

The present invention 10 could be a human skin lubricant or lubricating composition comprised of hydrophobic formulations that are obtained using silicone gels micronized in silicone fluids and a methodology 200 for the use of same. The formulations of the present invention 10 may be prepared by a variety of methods known in the art that generally incorporate homogenization or particle size reduction at one or more steps in the method of preparation of the invention. The preparation of the gels and their subsequent homogenization could further afford a convenient method of incorporating additional materials such as personal care ingredients or other active ingredients.

Compositions

One possible method of the making of the present invention 10 could involve: 1) the preparation of the silicone gel and addition or incorporation of personal care ingredients or active ingredients prior to homogenization or particle size reduction or 2) preparation of the silicone gel in the presence of personal care ingredients or active ingredients followed by homogenization.

As generally used herein, the phrase “silicone gel” could refer to any silicone containing material that may increase its volume upon substantial contact with a low molecular weight solvent that may or may not be volatile wherein the solvent diffuses into the silicone containing material.

The silicone gels as generally utilized in the formulations for the present invention 10 may be prepared in the presence of a variety of chemically appropriate solvents (hereinafter defined and listed). Once prepared, the silicone gels may be dispersed in a variety of chemically appropriate solvents (hereinafter defined and listed). In addition to the compositions and methodologies for the invention described below, a variety of the silicone gels for the invention 10 may also be prepared by condensation cure mechanisms, e.g. room temperature vulcanizable compositions.

Silicone Gel I.

The present invention 10 may utilize a silicone gel composition comprising: a first silicone and a second silicone (e.g., a lipophilic phase) having a viscosity below about 1,000 centistokes at 25.degrees. C. (hereinafter also referred to as dispersant medium or media.)

The first silicone could be formed by the hydrosilylation product of (1) a linear alkenyl polyorganosiloxane having the formula: M.sup.vi.sub.aD.sub.xD.sup.vi.sub.yM.sub.2-a where the subscript x is a number greater than 10, the subscript y is a number ranging from zero to about 20, the subscript a is a number ranging from 0 to 2, subject to the limitation that a+y is within the range of from 1 to about 20, with M.sup.vi defined as: R.sup.1R.sup.2R.sup.3SiO.sub.1/2 where R.sup.1 is a monovalent unsaturated hydrocarbon radical having from two to ten carbon atoms, and R.sup.2 and R.sup.3 are each independently one to eighty carbon atom monovalent hydrocarbon radicals, with D defined as: R.sup.4R.sup.5SiO.sub.2/2 where R.sup.4 and R.sup.5 are each independently one to eighty carbon atom monovalent hydrocarbon radicals, with D.sup.vi defined as: D.sup.vi=R.sup.6R.sup.7SiO.sub.2/2 where R.sup.6 is a monovalent unsaturated hydrocarbon radical having from two to ten carbon atoms, and R.sup.7 is independently a one to eighty carbon atom monovalent hydrocarbon radical with M defined as M=R.sup.8R.sup.9R.sup.10SiO.sub.1/2 with R.sup.8, R.sup.9, and R.sup.10 each independently a one to eighty carbon atom monovalent hydrocarbon radical; and (2) a hydride resin having the formula: (M.sup.H.sub.wQ.sub.z).sub.j where Q has the formula SiO.sub.4/2 and with M.sup.H defined as H.sub.bR.sup.11.sub.3-bSiO.sub.1/2 where R.sup.11 is a one to eighty carbon atom monovalent hydrocarbon radical where the subscript b is a number ranging from 1 to 3, with the subscripts w and z having a ratio of 0.5 to 4.0 respectively and the subscript j ranges from about 2.0 to about 100; wherein said hydrosilylation is conducted in the presence of (3) a hydrosilylation compatible solvent preferably a silicone having a viscosity below about 1,000 centistokes at 25.degrees. C. or a hydrosilylation compatible lipohilic phase (hereinafter also referred to as a hydrosilylation compatible solvent), thereby forming a gel.

The second silicone having a viscosity below about 1,000 centistokes at 25.degrees. C. (hereinafter also referred to as dispersant medium or media) wherein said hydrosilylation product is slurried in said lipophilic phase or said second silicone and subjected to mixing with said lipophilic phase or said second silicone; producing thereby a uniform mixture comprising said lipophilic phase or said second silicone and said hydrosilylation product whereby said uniform mixture has a viscosity ranging from 500 to 500,000 centistokes at 25.degrees. C.

The second silicone having a viscosity below about 1,000 centistokes at 25.degrees. C. is preferably selected from the group consisting of cyclic silicones having the formula: D.sub.f where the subscript f is an integer ranging from about 3 to about 6 with D defined as: R.sup.4R.sup.5SiO.sub.2/2 where R.sup.4 and R.sup.5 are each independently one to eighty carbon atom monovalent hydrocarbon radicals, the group of linear silicones having the formula: M′D′.sub.iM′ where D′ is defined as: R.sup.4R.sup.5SiO.sub.2/2 where R.sup.4 and R.sup.5 are each independently one to eighty carbon atom monovalent hydrocarbon radicals and M′ has the formula: R.sup.12R.sup.13R.sup.14SiO.sub.1/2 where R.sup.12, R.sup.13 and R.sup.14 are each independently one to eighty carbon atom monovalent hydrocarbon radicals and the group of branched silicones having the formula: T.sub.′D′M′.sub.″ where D′ and M′ are each independently selected and are as previously defined and T=R.sup.2SiO.sub.3/2, where R.sup.2 is independently selected and is as previously defined and the subscript ′ is at least 1, the subscript ″ ranges from 0 to 3′ and the subscript is 0 or positive with all three subscripts chosen so that the viscosity of T.sub.′D′M′.sub.″ is 1,000 centipose or less at 25.degrees. C.

Silicone Gel II.

Other gels suitable for the compositions of the present invention 10 may be comprised of the reaction products of an epoxy functional hydrido-siloxane molecule having the following formula: M.sub.alpha.M.sup.H.sub.beta.M.sup.E.sub.chi.D.sub.delta.D.sup.H.sub.- epsilon.D.sup.E..sub.phi.T.sub..gamma.T.sup.H.sub..eta.T.sup.E.sub.Q.sub.-kappa. where M=R.sup.1′R.sup.2′R.sup.3′SiO.sub.1/2; M.sup.H=R.sup.4′R.sup.5′HSiO.sub.1/2; M.sup.E=R.sup.6′R.sup.7′R.sup.ESiO.sub.1/2; D=R.sup.8′R.sup.9′SiO.sub.2/2; D.sup.H=R.sup.10′HSiO.sub.2/2; D.sup.E=R.sup.11′R.sup.ESiO.sub.2/2; T=R.sup.12′SiO.sub.3/2; T.sup.H=HSiO.sub.3/2; T.sup.E=R.sup.ESiO.sub.3/2; and Q=SiO.sub.4/2; where R.sup.1′, R.sup.2′, R.sup.3′, R.sup.8′, R.sup.9′ and R.sup.12′ are independently monovalent hydrocarbon radicals having from one to sixty carbon atoms; R.sup.4′, R.sup.5′ and R.sup.10′ are independently monovalent hydrocarbon radicals having from one to sixty carbon atoms or hydrogen; R.sup.6′, R.sup.7′, R.sup.11′ are independently monovalent hydrocarbon radicals having from one to sixty carbon atoms or R.sup.E; each R.sup.E is independently a monovalent hydrocarbon radical containing one or more oxirane moieties having from one to sixty carbon atoms; the stoichiometric subscripts .alpha., .beta., .chi., .delta., .epsilon., .phi., .gamma., .eta., , and .kappa. are either zero or positive subject to the following limitations: alpha.+.beta.+.chi.>1; beta.+.epsilon.+.eta.>1; chi.+.phi.+>1; .beta.+.epsilon.+.eta.>.chi.+.phi.+; and when delta.+.epsilon.+.phi.+.gamma.+.eta.++.kappa.=0, .alpha.+.beta.+.chi.=2.

The reaction product of an epoxy functional hydrido siloxane molecule is preferably prepared in an epoxy gel formation medium selected from a lipophilic phase or a silicone fluid selected from the group consisting of cyclic silicones having the formula: D.sub.f where the subscript f is an integer ranging from about 3 to about 6 with D defined as: R.sup.4R.sup.5SiO.sub.2/2 where R.sup.4 and R.sup.5 are each independently one to eighty carbon atom monovalent hydrocarbon radicals, the group of linear silicones having the formula: M′D′.sub.iM′ where D′ is defined as: R.sup.4R.sup.5SiO.sub.2/2 where R.sup.4 and R.sup.5 are each independently one to eighty carbon atom monovalent hydrocarbon radicals and M′ has the formula: R.sup.12R.sup.13R.sup.14SiO.sub.1/2 where R.sup.12, R.sup.13 and R.sup.14 are each independently one to eighty carbon atom monovalent hydrocarbon radicals and the group of branched silicones having the formula: T.sub.′D′M′.sub.″ where D′ and M′ are each independently selected and are as previously defined and T=R.sup.2SiO.sub.3/2, where R.sup.2 is independently selected and is as previously defined and the subscript ′ is at least 1, the subscript ″ ranges from 0 to 3′ and the subscript is 0 or positive with all three subscripts chosen so that the viscosity of T.sub.′D′M′.sub.″ is 1,000 centipose or less at 25.degrees. C.

Once prepared, the type II silicone gels that may be utilized in the composition of the present invention 10 may be slurried and mixed in a dispersant medium selected from a lipophilic phase or a silicone selected from the group consisting of cyclic silicones having the formula D.sub.f where the subscript f is an integer ranging from about 3 to about 6 with D defined as: R.sup.4R.sup.5SiO.sub.2/2 where R.sup.4 and R.sup.5 are each independently one to eighty carbon atom monovalent hydrocarbon radicals, the group of linear silicones having the formula: M′D′.sub.iM′ where D′ is defined as: R.sup.4R.sup.5 SiO.sub.2/2 where R.sup.4 and R.sup.5 are each independently one to eighty carbon atom monovalent hydrocarbon radicals and M′ has the formula: R.sup.12R.sup.13R.sup.14SiO.sub.1/2 where R.sup.12, R.sup.13 and R.sup.14 are each independently one to eighty carbon atom monovalent hydrocarbon radicals and the group of branched silicones having the formula: T.sub.′D′M′.sub.″ where D′ and M′ are each independently selected and are as previously defined and T=R.sup.2SiO.sub.3/2, where R.sup.2 is independently selected and is as previously defined and the subscript ′ is at least 1, the subscript ″ ranges from 0 to 3′ and the subscript is 0 or positive with all three subscripts chosen so that the viscosity of T.sub.′D′M′.sub.″ is 1,000 centipose or less at 25.degrees. C.

Silicone Gel III.

The present invention 10 may be formed from a silicone gel composition comprising: (A) a silicone formed by the hydrosilylation product of (1) a linear hydrogen polyorganosiloxane having the formula: M.sup.H.sub.aD.sub.xD.sup.H.sub.yM.sub.2-a where the subscript x is a number greater than 10, the subscript y is a number ranging from zero to about 20, the subscript a is a number ranging from 0 to 2, subject to the limitation that a+y is within the range of from 1 to about 20, with M.sup.H defined as: R.sup.1R.sup.2R.sup.3SiO.sub.1/2 where R.sup.1 is hydrogen, R.sup.2 and R.sup.3 are each independently one to eighty carbon atom monovalent hydrocarbon radicals, with D defined as: R.sup.4R.sup.5SiO.sub.2/2 where R.sup.4 and R.sup.5 are each independently one to eighty carbon atom monovalent hydrocarbon radicals, with D.sup.H defined as: D.sup.H=R.sup.6R.sup.7SiO.sub.2/2 where R.sup.6 is hydrogen and R.sup.7 is independently a one to eighty carbon atom monovalent hydrocarbon radical with M defined as M=R.sup.8R.sup.9R.sup.10SiO.sub.1/2 with R.sup.8, R.sup.9, and R.sup.10 each independently a one to eighty carbon atom monovalent hydrocarbon radical; and (2) an alkenyl resin having the formula: (M.sup.vi.sub.wQ.sub.z).sub.j where Q has the formula SiO.sub.4/2 and with M.sup.vi defined as R.sup.11.sub.bR.sup.12.sub.3-bSiO.sub.1/2 where R.sup.11 is a monovalent unsaturated hydrocarbon radical having from two to ten carbon atoms, R.sup.12 is a one to eighty carbon atom monovalent hydrocarbon radical where the subscript b is a number ranging from 1 to 3, with the subscripts w and z having a ratio of 0.5 to 4.0 respectively and the subscript j ranges from about 2.0 to about 100; wherein said hydrosilylation is conducted in the presence of (3) a hydrosilylation compatible solvent preferably a silicone having a viscosity below about 1,000 centistokes at 25.degrees. C. or a hydrosilylation compatible lipohilic phase (hereinafter also referred to as hydrosilylation compatible solvent), thereby forming a gel; and (B) a lipophilic phase or a second silicone having a viscosity below about 1,000 centistokes at 25.degrees. C. (hereinafter also referred to as dispersant medium or media) wherein said hydrosilylation product is slurried in said lipophilic phase or said second silicone and subjected to mixing with said lipophilic phase or said second silicone; producing thereby a uniform mixture comprising said lipophilic phase or said second silicone and said hydrosilylation product whereby said uniform mixture has a viscosity ranging from 500 to 500,000 centistokes at 25.degrees. C.

Silicone Gel IV

A first silicone being the hydrosilylation reaction product of a linear alkenyl organopolysiloxane (as previously defined) having two or more alkenyl functionalities per molecule as above with a linear hydrogen organopolysiloxane (as previously defined) having two or more hydrogen functionalities per molecule prepared as above in the presence of a hydrosilylation compatible solvent or silicone, D.sub.f, M′D′.sub.iM′ or T.sub.′D′M′.sub.″ where D.sub.f, M′D′.sub.iM′ and T.sub.′D′M′.sub.″ are as previously defined. The gel as prepared may then be slurried with a lipophilic phase or a silicone having a viscosity below about 1,000 centistokes at 25.degrees. C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25.degrees. C.

Silicone Gel V

A first silicone being the hydrosilylation reaction product of an alkenyl organopolysiloxane resin having two or more alkenyl functionalities per molecule having the formula M.sup.vi.sub.cD.sub.dD.sup.vi.sub.eT.sub.gT.sup.vi.sub.kM.sub.2-cM″.sub.- g+k with a hydrogen organopolysiloxane resin having two or more hydrogen functionalities per molecule having the formula M.sup.H.sub.nD.sub.pD.sup.H.sub.rT.sub.sT.sup.H.sub.uM.sub.vM″.sub.s+u where all the terms are as previously defined with T=R.sup.16SiO.sub.3/2 where R.sup.16 is a one to eighty carbon atom monovalent hydrocarbon radicals; T.sup.vi=R.sup.17SiO.sub.3/2 where R.sup.17 is a monovalent unsaturated hydrocarbon radical having from two to forty carbon atoms; T.sup.H=HSiO.sub.3/2; M″ is independently M.sup.H, M.sup.vi or M and the subscripts c, d, e, g, k, n, p, r, s, u, and v are either zero or positive subject to the limitations that g+k+s+u is .gtoreq.1; c+e+k.gtoreq.2 and n+r+u.gtoreq.2; prepared in a hydrosilylation compatible solvent and slurried in a lipophilic phase or a second silicone having a viscosity below about 1,000 centistokes at 25.degrees. C. (hereinafter also referred to as dispersant medium or media or low viscosity silicone fluid) wherein said hydrosilylation product is slurried in said lipophilic phase or said second silicone and subjected to mixing with said lipophilic phase or said second silicone; producing thereby a uniform mixture comprising said lipophilic phase or said second silicone and said hydrosilylation product whereby said uniform mixture has a viscosity ranging from 500 to 500,000 centistokes at 25.degrees. C.

Silicone Gel VI

A first silicone being the reaction product of a linear hydrido organopolysiloxane having two or more hydride functionalities per molecule with an .alpha., .omega. reactive organic molecule possessing two or more reactive functionalities per molecule in the presence of a lipophilic phase or second silicone, D.sub.f, M′D′.sub.iM′ or T.sub.′D′M′.sub.″ where D.sub.f, M′D′.sub.iM′ and T.sub.′D′M′.sub.″ are as previously defined. The reactive functionalities of the .alpha., .omega. reactive organic molecule possessing two or more functionalities per molecule are selected from the group of organic functional groups consisting of olefins, acetylenes, vinylethers, acrylates or acrylate esters (e.g. CH2═CHCOOROCOCH═CH2), and alcohols and the like. Thus the .alpha., .omega. reactive organic molecule possessing two or more functionalities per molecule subtends a large group of organic molecules that includes .alpha., .omega.-di-olefins, .alpha., .omega.-olefins possessing a polyolefinic functionality, .alpha., .omega.-di-acetylenes, .alpha., .omega.-di-acetylenes possessing a polyacetylenic functionality, including side chain substituted variations where the side chains possess reactive functionality as herein defined. This gel is prepared in a hydrosilylation compatible solvent and slurried in a lipophilic phase or a silicone having a viscosity below about 1,000 centistokes at 25.degrees. C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25.degrees. C.

Silicone Gel VII

Other gels suitable for the compositions of the present invention 10 comprise the reaction products of a vinyl functional hydrido-siloxane molecule having the following formula: M.sub.a′M.sup.vi.sub.b′M.sup.H.sub.c′D.sub.d′D.sup.vi.sub.e′D.sup.H.sub.f- T.sub.g′T.sup.vi.sub.h′T.sup.H.sub.iQ.sub.j′ where all the terms are as previously defined and the subscripts a′, b′, c′, d′, e′, f′, g′, h′, i′ and j′ are either 0 or a positive integer for well defined molecular species subject to the limitation b′+e′+h′ is greater than or equal to one and further subject to the limitation that c′+f+i′ is greater than or equal to one. Mixtures of compounds where individual molecular species possess the formula, M.sub.a′M.sup.vi.sub.b′M.sup.H.sub.c′D.sub.d′D.sup.vi.sub.e′D.sup.H.sub.f- T.sub.g′T.sup.vi.sub.h′T.sup.H.sub.iQ.sub.j′, will analyze for non-integral values of the subscripts because of the fact that it is a mixture and not a pure compound. Thus for mixtures of compounds possessing the formula, M.sub.a′M.sup.vi.sub.b′M. sup.H.sub.c′D.sub.d′D.sup.vi.sub.e′D.sup.H.sub.f- T.sub.g′T.sup.vi.sub.h′T.sup.H.sub.i′Q.sub.j′, the subscripts a′, b′, c′, d′, e′, f, g′, h′, i′and j′ will be zero or positive. Compounds possessing the formula M.sub.a′M.sup.vi.sub.b′M.sup.H.sub.c′D.sub.d′D.sup.vi.sub.e′D.sup.H.sub.f- T.sub.g′T.sup.vi.sub.h′T.sup.H.sub.i′Q.sub.j′ may be prepared by the procedures and methods disclosed in U.S. Pat. Nos. 5,698,654; 5,753,751; and 5,965,683 herewith specifically incorporated by reference. These materials may be reacted with the silicone precursors to the class II silicone gels previously defined herein or they may self-react in the presence of a noble metal hydrosilylation catalyst as is known in the art. These materials are prepared in a hydrosilyation compatible solvent and slurried in a lipophilic phase or a silicone having a viscosity below about 1,000 centistokes at 25.degrees. C. (hereinafter also referred to as dispersant medium or media) wherein said hydrosilylation product is slurried in said lipophilic phase or said silicone and subjected to mixing with said lipophilic phase or said silicone; producing thereby a uniform mixture comprising said lipophilic phase or said silicone and said hydrosilylation product whereby said uniform mixture has a viscosity ranging from 500 to 500,000 centistokes at 25.degrees. C.

Many types of noble metal catalysts for hydrosilylation (or SiH olefin addition reaction) are known and such noble metal catalysts may be used for the preparative reactions involved in making the compositions of the present invention 10. The most preferred noble metals are those of the platinum group metals, specifically rhodium and platinum. When optical clarity of the resulting addition product is required the preferred catalysts are those catalysts that are compounds that are soluble in the reaction mixture. One such platinum compound can be selected from those having the formula (PtCl.sub.2Olefin) and H(PtCl.sub.3Olefin). The olefin shown in the previous two catalyst compound formulas can be almost any type of olefin but is preferably an alkenylene having from 2 to 8 carbon atoms, a cycloalkenylene have from 5 to 7 carbon atoms or styrene. Specific olefins utilizable in the above formulas are ethylene, propylene, the various isomers of butylene, octylene, cyclopentene, cyclohexene, cycloheptene, and the like.

A further platinum containing material usable in the compositions of the present invention 10 is the cyclopropane complex of platinum chloride.

Further, the platinum containing material can be a complex formed from chloroplatinic acid with up to 2 moles per gram of platinum of a member selected from the class consisting of alcohols, ethers, aldehydes and mixtures of the above.

Persons skilled in the art can easily determine an effective amount of noble metal or platinum catalyst. Generally, an effective amount ranges from about 0.1 to 50 parts per million of the total organopolysiloxane composition.

The human skin lubricating compositions of the present invention 10 are prepared in general by preparing the gels herein described, dispersing the gels in a dispersing medium and homogenizing the dispersed gels having a particulate matter size ranging from 0.2 to 50 microns. The human skin lubricating compositions of the present invention 10 could also be described as being ranges of percentages by weight. For example, the invention being a gel formed from cross-linking siloxane polyethers, the gel being further micronized in one or more low viscosity, silicone fluids, wherein the siloxane polyethers are cross-linked by combining a low viscosity organopolysiloxane, an unsaturated organopolysiloxane, and a Si—H functional cross-linker. The low viscosity organopolysiloxane being present in an amount of 35% to 90% by weight; the unsaturated organopolysiloxane is present in an amount of about 5% to about 75% by weight, and the Si—H functional cross-linker is present in an amount of about 1-10% by weight. In another embodiment of the invention, the low viscosity organopolysiloxane is present in an amount of 10% to 80% by weight; the unsaturated organopolysiloxane is present in an amount of about 10% to about 90% by weight, and the Si—H functional cross-linker is present in an amount of about 1-10% by weight.

Preparing the Skin Lubricant with Personal Care Ingredients or Active Ingredients

Additional personal care ingredients or active ingredients may be added to the gels and the dispersant medium to impart required or desired additional characteristics to the invention 10.

Methodologies of preparing the present invention 10 could comprise one method of adding personal care ingredients or active ingredients to the gel precursors and forming the gels in the presence of personal care ingredients or active ingredients, dispersing the resultant gel in a dispersant medium and homogenizing the gel, the pigments, personal care ingredients or active ingredients, and the dispersant medium. Another method of preparing the present invention 10 could comprise of preparing the gel, adding thereto personal care ingredients or active ingredients either before or after dispersing the gel in a dispersant medium followed by homogenization. Yet another method of preparing the present invention 10 could entail preparing the gel, dispersing it in a dispersant medium, homogenizing the gel in the dispersant medium and adding thereto personal care ingredients or active ingredients.

The gels of the present invention 10 are prepared either in a hydrosilylation compatible medium or solvent or an epoxy-gel formation compatible medium or solvent depending on the chemical nature of the gel being prepared. Both classes of preparation media include silicone solvents, preferably a silicone selected from the group of cyclic silicones having the formula D.sub.f where the subscript f is an integer ranging from about three to about 6 with D defined as R.sup.4R.sup.5SiO.sub.2/2 where R.sup.4 and R.sup.5 are each independently one to eighty carbon atom monovalent hydrocarbon radicals; the group of linear silicones having the formula M′D′.sub.iM′ where D′ is defined as R.sup.4R.sup.5SiO.sub.2/2 where R.sup.4 and R.sup.5 are each independently one to eighty carbon atom monovalent hydrocarbon radicals and M′ has the formula R.sup.12R.sup.13R.sup.14SiO.sub.1/2 where R.sup.12, R.sup.13 and R.sup.14 are each independently one to eighty carbon atom monovalent hydrocarbon radicals; and the group of branched silicones having the formula: T.sub.′D′M′.sub.″ where D′ and M′ are each independently selected and are as previously defined and T=R.sup.2SiO.sub.3/2, where R.sup.2 is independently selected and is as previously defined and the subscript ′ is at least 1, the subscript ″ ranges from 0 to 3′ and the subscript is 0 or positive with all three subscripts chosen so that the viscosity of T.sub.′D′M′.sub.″ is 1,000 centipose or less at 25.degrees. C.

The hydrosilylation compatible medium or solvent are selected from the group consisting of silicones and substituted silicones including: silicone oils of the desired viscosity from D.sub.4 to 10,000 cps oils; polyethersilicone copolymers where the polyethers vary from 200 to 3000 molecular weight and may consist of alkylene oxide chains based on one, two or more types of monomer units such as ethylene oxide, propylene oxide or butylene oxide and may be attached to the silicone with 1 to six carbon chain, or through an silicone oxygen bond; polyester silicone copolymers; alkyl, aromatic or alkylaromatic substituted siloxanes; alkoxy substituted siloxanes including: substituted methoxy, ethoxy, propoxy, octyloxy, dodecanoxy, cetyryloxy or isostearyloxy siloxanes or other organically substituted siloxanes or siloxanes containing multiple organic substituents that are compatible with hydrosilylation reactions; hydro carbon solvents including: tetradecane, isododecane, isohexadecane, mineral oil, hydrogenate polydecene, apricot oil; ester solvents including: isopropyl myristate, diisopropyl adipate, isodecyl neopentanoate; ethers including: PPG-14 butyl ether, PPG 3 myristyl ether, ethoxylated alkylphenols; glyceryl esters of fatty acids including: sunflower oils, caprylic/capric triglyceride, C.sub.10-18 triglyceride; fatty acid glycerides including: glyceryl stearate, glyceryl dioleate; non-volatile fluorinated oil including: fluorinated silicones and fluorinated esters; aromatic solvents including; benzene, toluene and alkylbenzenes; and alcohols including: isopropanol, octanol, dodecanol, hexadecanol, cetearyl alcohol, isostearyl alcohol, myristyl alcohol.

The epoxy gel formation compatible medium or solvent is primarily defined by solvent inertness and is preferably selected from the group of silicone solvents D.sub.f, M′D′.sub.iM′ or T.sub.′D′M′.sub.″ where D.sub.f, M′D′.sub.iM′ and T.sub.′D′M′.sub.″ are as previously defined and hydrocarbon solvents selected from the group consisting of paraffinic, iso-paraffinic, aromatic and alkyl aromatic solvents.

The compositions according to the present invention 10 therefore advantageously comprise a stable dispersion of particles of at least one silicone in a dispersant medium preferably, a lipophilic phase, the lipophilic phase preferably selected from the group consisting of 1) physiologically acceptable liquid lipophilic or fatty phases and 2) silicone fluids selected from the group consisting of D.sub.f, M′D′.sub.iM′ or T.sub.′D′M′.sub.″ where D.sub.f, M′D′D′.sub.iM′ and T.sub.′D′M′.sub.″ are as previously defined.

These dispersions may in particular be provided in the form of nanoparticles of silicone gel in a stable dispersion in the said lipophilic or fatty phase. The nanoparticles are preferably of between 5 and 600 nm in size, given that above about 600 nm the dispersions of particles become much less stable. This size range includes all specific values and sub-ranges there-between, including 10, 25, 50, 100, 200, 300, 400 and 500 nm.

The liquid lipophilic or fatty phase in which the siloxane or silicone polymer may be dispersed may consist of any dermatologically acceptable, and more generally physiologically acceptable, oil chosen in particular from oils of inorganic, animal, plant or synthetic origin, carbonaceous oils, hydrocarbon oils, fluorinated oils and/or silicone oils, alone or in the form of a mixture insofar as they form a homogeneous and stable mixture and are compatible with the use envisaged. “Liquid fatty phase” refers to any nonaqueous medium which is liquid at room temperature. “Volatile fatty phase” refers to any nonaqueous medium capable of evaporating from the skin or the lips, at room temperature, in less than one hour.

Nonvolatile liquid lipophilic or fatty phase which can be used in the invention, include hydrocarbon oils such as paraffin oil or liquid petroleum jelly, vison oil, turtle oil, soya bean oil, perhydrosqualene, sweet almond oil, calophyllum oil, palm oil, grapeseed oil, sesame oil, maize oil, parleam oil, arara oil, rapeseed oil, sunflower oil, cottonseed oil, apricot oil, castor oil, avocado oil, jojoba oil, olive oil or cereal germ oil; esters of lanolic acid, oleic acid, lauric acid or stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol or oleyl alcohol, linoleyl or linolenyl alcohol, isostearyl alcohol or octyl dodecanol; silicone oils such as polydimethylsiloxane (PDMS), which are optionally phenylated, such as phenyl trimethicones, or which are optionally substituted with optionally fluorinated aliphatic and/or aromatic groups, or with functional groups such as hydroxyl, thiol and/or amine groups; polysiloxanes modified with fatty acids, fatty alcohols or polyoxyalkylenes, fluorinated silicones and perfluorinated oils.

One or more oils which are volatile at room temperature and atmospheric pressure may optionally be used. These volatile oils have for example a steam pressure at ambient temperature and pressure of, preferably, from 1.times.10.sup.-3 to 300 mm Hg, provided that the boiling point is greater than 25.degrees. C. These volatile oils facilitate in particular the application of the composition to the skin, the mucous membranes and the superficial body growths. These oils may be hydrocarbon oils, silicone oils optionally comprising alkyl or alkoxy groups at the end of the silicone or pendant chain.

The volatile silicone oil which may be used in the invention, is selected from the group consisting of D.sub.f, M′D′.sub.iM′ or T.sub.′D′M′.sub.″ where D.sub.f, M′D′.sub.iM′ and T.sub.′D′M′.sub.″ as previously defined. The volatile oils represent preferably from 0 to 97.5% of the total weight of the composition, and more preferably from 5 to 85%. These ranges include all specific values and sub-ranges there-between, including 0.5, 1, 2, 8, 10, 15, 25, 30, 50, 60, 70, 80, 90 and 95% by weight.

Among the liquid lipophilic or fatty phases suitable for the compositions of the present invention 10 are vegetable oils formed by esters of fatty acids and polyols, in particular triglycerides, such as sunflower, sesame or rapeseed oil, or the esters derived from long-chain acids or alcohols (that is to say having from 6 to 20 carbon atoms), in particular the esters of formula RCOOR′ in which R represents the residue of a higher fatty acid containing from 7 to 19 carbon atoms and R′ represents a hydrocarbon chain containing from 3 to 20 carbon atoms, such as palmitates, adipates and benzoates, in particular diisopropyl adipate. There may also be mentioned the hydrocarbons and in particular paraffin oils, liquid petroleum jelly, or hydrogenated polyisobutylene, isododecane, or alternatively the “ISOPARs”, volatile isoparaffins. There may also be mentioned the silicone oils such as polydimethylsiloxanes and polymethylphenylsiloxanes, optionally substituted with optionally fluorinated aliphatic and/or aromatic groups, or with functional groups such as hydroxyl, thiol and/or amine groups, and the volatile, in particular cyclic, silicone oils. There may also be mentioned the solvents, alone or in the form of a mixture, chosen from (i) linear, branched or cyclic esters having more than 6 carbon atoms, (ii) ethers having more than 6 carbon atoms, (iii) ketones having more than 6 carbon atoms. Monoalcohols having an overall solubility parameter according to the HANSEN solubility space of less than or equal to 20 (MPa).sup.1/2 are understood to mean the aliphatic fatty alcohols having at least 6 carbon atoms, the hydrocarbon chain containing no substitution group. As mono-alcohols according to the invention, there may be mentioned oleyl alcohol, decanol, dodecanol, octadecanol, and linoleyl alcohol.

Preferably the dispersant is selected from the group consisting of hydrocarbon oils, paraffin oil, liquid petroleum jelly, vison oil, turtle oil, soya bean oil, perhydrosqualene, sweet almond oil, calophyllum oil, palm oil, grapeseed oil, sesame oil, maize oil, parleam oil, arara oil, rapeseed oil, sunflower oil, cottonseed oil, apricot oil, castor oil, avocado oil, jojoba oil, olive oil, cereal germ oil; esters of lanolic acid, esters of oleic acid, esters of lauric acid, esters of stearic acid; isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate, diglyceryl triisostearate, myristic acid, palmitic acid, stearic acid, behenic acid, oleic acid, linoleic acid, linolenic acid, isostearic acid; cetanol, stearyl alcohol, oleyl alcohol, linoleyl or linolenyl alcohol, isostearyl alcohol or octyl dodecanol; silicone oils, polydimethylsiloxane, phenylated polydimethylsiloxane, polymethylphenylsiloxanes, phenyl trimethicones, phenyl trimethicones substituted with fluorinated aliphatic and/or aromatic groups, phenyl trimethicones substituted with functional groups such as hydroxyl, thiol and/or amine groups; polysiloxanes modified with fatty acids, fatty alcohols or polyoxyalkylenes; fluorinated silicones, perfluorinated oils, vegetable oils, sunflower oil, sesame oil, rapeseed oil, the esters long-chain acids or alcohols having the formula RCOOR in which R represents the residue of a higher fatty acid containing from 7 to 19 carbon atoms and R′ represents a hydrocarbon chain containing from 3 to 20 carbon atoms, hydrogenated polyisobutylene, isododecane, volatile isoparaffins, oleyl alcohol, decanol, dodecanol, octadecanol and linoleyl alcohol.

The choice of the non-aqueous medium is made by persons skilled in the art as a function of the nature of the monomers constituting the polymer and/or of the nature of the stabilizer, as indicated below. In particular, it is possible to use a polar or weakly polar oil such as vegetable oils of the long carbon chain-containing triglyceride type (apricot oil, jojoba oil) or the long carbon chain-containing esters such as octyldodecyl neopentanoate, the alkanes such as parleam oil, and the silicone oils. Furthermore, the total liquid lipophilic or fatty phase in which the polymer is dispersed may represent from 30% to 95% of the total weight of the composition and preferably from 30 to 75%. These ranges for the total liquid lipophilic or fatty phase include all specific values and sub-ranges there-between, including 35, 40, 45, 50, 60, 70, 80, 85, 90 and 95% of the total weight of the composition. The nonvolatile part represents at least 0.5% and in practice from 1 to 35% of the total weight of the composition. These ranges for the nonvolatile part include all specific values and sub-ranges there-between, including 2, 3, 5, 10, 15, 20 and 25% of the total weight of the composition.

In numerous preferred embodiments, the human skin lubricating composition of the present invention 10 may further comprise one or more personal care ingredients. Suitable personal care ingredients or active ingredients include, for example, emollients, moisturizers, humectants, pigments, including pearlescent pigments such as, for example, bismuth oxychloride and titanium dioxide, fragrances, biocides, preservatives, antioxidants, anti-microbial agents, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, solid or liquid silicone resins, silicone quats, organic oils, waxes, film formers, thickening agents such as, for example, fumed silica or hydrated silica, particulate fillers, such as for example, talc, kaolin, starch, modified starch, teflon, nylon, polymethylsilsequioxane, clays, such as, for example, bentonite and organo-modified clays.

Suitable human skin lubricating compositions are made by combining, in a manner known in the art, such as, for example, by mixing, one or more of the above components with the siloxane copolymer network or precursors thereto, preferably in the form of the silicone compositions of the present invention 10. Thus the entrapped, occluded or encapsulated materials may be incorporated into the silicone network at any point in the preparation of the lubricating compositions of the present invention 10 provided they do not interfere with the preparation of the silicone gel or siloxane copolymer network.

The compositions of the present invention 10 may be utilized as prepared or as one or more components in non-aqueous emulsions. As is generally known, emulsions comprise at least two immiscible phases one of which is continuous and the other which is discontinuous. Further emulsions may be liquids with varying viscosities comprising solids. Additionally the particle size of the emulsions may render them microemulsions and when sufficiently small such microemulsions may be transparent. Further it is also possible to prepare emulsions of emulsions and these are generally known as multiple emulsions.

These primary types of emulsions may be non-aqueous emulsions where the discontinuous phase comprises a non-aqueous hydroxylic solvent and the continuous phase comprises the polyether siloxane copolymer network of the present invention 10; and non-aqueous emulsions where the continuous phase comprises a non-aqueous hydroxylic organic solvent and the discontinuous phase comprises the polyether siloxane copolymer network of the present invention 10.

As used herein the term “non-aqueous hydroxylic organic compound” means hydroxyl containing organic compounds as exemplified by but not limited to alcohols, glycols, polyhydric alcohols and polymeric glycols and mixtures thereof that are liquid at room temperature, e.g. about 25.degrees. C., and about one atmosphere pressure. The non-aqueous organic hydroxylic solvents are selected from the group consisting of hydroxyl containing organic compounds comprising alcohols, glycols, polyhydric alcohols and polymeric glycols and mixtures thereof that are liquid at room temperature, e.g. about 25.degrees. C., and about one atmosphere pressure. Preferably the non-aqueous hydroxylic organic solvent is selected from the group consisting of ethylene glycol, ethanol, propyl alcohol, iso-propyl alcohol, propylene glycol, dipropylene glycol, tripropylene glycol, butylene glycol, iso-butylene glycol, methyl propane diol, glycerin, sorbitol, polyethylene glycol, polypropylene glycol mono alkyl ethers, polyoxyalkylene copolymers and mixtures thereof.

Yet another skin lubrication composition of the invention 10 could comprise of a mono-oxyalkylene-functional silicone having at least one carbinol functional group, such as Dow Corning® 5562 Carbinol Fluid. This composition could also further include one or more personal care ingredients or active ingredients as discussed above. The composition could be generally applied to the desired portion of skin to be lubricated with out further processing.

### Sample Compositions

### Sample 1

Skin lubrication gel #1 was prepared as follows: 250 parts of GE-Silicone product SF839 heavy gel (polyether siloxane cross-linked) was added to 750 parts of GE-Silicone fluid SF-1202 (decamethylcyclo-pentasiloxane) and mixed with a Gaulin homogenizer at 8000 psi pressure for 1 hour at 150 degrees Fahrenheit.

### Example 2

Skin lubrication gel #2 was prepared as follows: 350 parts of GE-Silicone product SF-839 heavy gel (polyether siloxane cross-linked) was added to 650 parts of GE-Silicone fluid SF-1202 (decamethylcyclo-pentasiloxane) and mixed with a Gaulin homogenizer at 8000 psi pressure for 1 hour at 150 degrees Fahrenheit.

### Example 3

Skin lubrication gel #3 was prepared as follows: 450 parts of GE-Silicone product SF-839 heavy gel (polyether siloxane cross-linked) was added to 550 parts of GE-Silicone fluid SF-1202 (decamethylcyclo-pentasiloxane) and mixed with a Gaulin homogenizer at 8000 psi pressure for 1 hour at 150 degrees Fahrenheit.

### Example 4

Skin lubrication gel #4 was prepared as follows: 550 parts of GE-Silicone product SF-839 heavy gel (polyether siloxane cross-linked) was added to 450 parts of GE-Silicone fluid SF-1202 (decamethylcyclo-pentasiloxane) and mixed with a Gaulin homogenizer at 8000 psi pressure for 1 hour at 150 degrees Fahrenheit.

### Example 5

Skin lubrication gel #5 was prepared as follows: 650 parts of GE-Silicone product SF-839 heavy gel (polyether siloxane cross-linked) was added to 350 parts of GE-Silicone fluid SF-1202 (decamethylcyclo-pentasiloxane) and mixed with a Gaulin homogenizer at 8000 psi pressure for 1 hour at 150 degrees Fahrenheit.

### Example 6

Skin lubrication gel #6 was prepared as follows: 250 parts of GE-Silicone product SF-839 heavy gel (polyether siloxane cross-linked) was added to 750 parts of GE-Silicone fluid SF-96-5 (low viscosity dimethicone) and mixed with a Gaulin homogenizer at 8000 psi pressure for 1 hour at 150 degrees Fahrenheit.

### Example 7

Skin lubrication gel #7 was prepared as follows: 350 parts of GE-Silicone product SF-839 heavy gel (polyether siloxane cross-linked) was added to 550 parts of GE-Silicone fluid SF-96-5 (low viscosity dimethicone) and mixed with a high speed homo-mixer (6000 rpm) for 1 hour at 150 degrees Fahrenheit. Then 50 parts of octyl salicylate and 50 parts of methol was added and mixed for another 15 minutes.

### Example 8

Skin lubrication gel #7 was prepared as follows: 450 parts of GE-Silicone product SF-839 heavy gel (polyether siloxane cross-linked) was added to 450 parts of GE-Silicone fluid SF-96-5 (low viscosity dimethicone) and mixed with a high speed homo-mixer (6000 rpm) for 1 hour at 150 degrees Fahrenheit. Then 80 parts of powdered cross-linked gelatin and 10 parts of lemon-grass oil was added and mixed for another 15 minutes.

These samples are to be construed as exemplary in nature only and are not intended in any way to limit the appended claims. While these examples have shown one particular order of adding the active ingredients and various components to produce the human skin lubrication compositions of the present invention 10, it is contemplated that other orders of addition may produce different benefits. The order of addition explicitly taught herein is not to be interpreted as the exclusive manner of producing the mixtures that represent the compositions that can be produced by the techniques of the present invention 10. Such different orders of addition could be, for example, adding active ingredients, e.g. personal care ingredients, prior to homogenization or particle size reduction of the copolymer network or alternatively adding such active ingredients after homogenization. It is contemplated that a person having ordinary skill in the art would be able to produce obvious variations of the subject matter and disclosures herein contained that would be by reason of such ordinary skill within the literal or equitable scope of the appended claims.

Methodology of Use

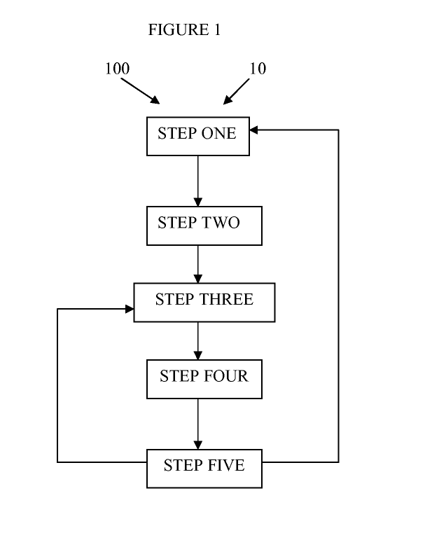

As substantially shown in FIG. 1, one possible methodology for the use of the present invention could be a process 100, which could start with step one, the preparation. In step one, the composition of the present invention 10 could be prepared in the various manners as set forth above using the above-described constituents. At the substantial completion of step one, the process 100 could proceed to step two, application.

In step two, application, the operator of the present invention 10 could select what flexible and elastic skin contact article(s) (e.g., compression wear, latex examination glove and the like) is (are) to be applied and determine which portion of the body the selected article is to be applied. The operator could then determine how much of the composition is to be used and then apply that amount to the skin of the selected portion of the body. This application of the composition could be accomplished by cupping a hand, placing the desired amount in the palm of the hand, then rubbing the palm upon the selected portion of the body (e.g., hands, limbs, feet, and the like.) In this manner, the application of the composition of the present invention 10 upon the selected portion could effectively reduce the selected portion's surface frictional coefficient as caused by the presence of oils, moisture, and the like. At the substantial completion of step two, the process 100 could proceed to step three, contacting the selected portion.

In step three, contacting the selected portion, the operator could otherwise grasp the chosen flexible and elastic skin contact article (e.g., latex examination glove) and manipulate it into position to contact some aspect of that portion of human body to which the invention 10 was applied. As step three is substantially completed, the process 100 could proceed to step four, moving the article relative to the portion.

In step four, moving the article relative to the portion, the operator could move the elastic and flexible skin contact article (e.g., examination glove, compression wear, and the like) to the lubricated portion of the human skin (e.g., the operator could move the selected portion within the selected article) to generally allow at least a partial encapsulation of the portion within at least a part of the article. The application of the composition of the present invention 10 may generally reduce the friction characteristic(s) of the skin's surface caused by the presence of any moisture and oil presence upon the skin surface, thus allowing movement of the portion within the article to take place with less force and exertion by the user. Such movement could include at least a partial encapsulation at least a portion of a lubricated hand within a latex glove; a least a lubricated portion of a limb within compression wear; and the like. At the conclusion of step four, the process could proceed to step five, removal.

At step five, removal, the operator, when needed, could then remove the article from the lubricated portion of the skin. The invention's previously-described friction reduction qualities could also possibly reducing the amount of effort and force generally required to remove the article from the lubricated portion even though more sweat and oils may have accumulated on the lubricated skin during the wearing of the article. When the removed article needs to be redonned upon the lubricated portion then the process 100 could proceed to step three. If the selected article or different article needs to be fitted to a different unlubricated portion of the body, then the process 100 could proceed back to step one.

### CONCLUSION

As generally shown above, the present invention may provide a skin lubricant that may be comprised of a low molecular weight polymeric silicone gel that is sheared and then mixed into one or more silicone fluids or may utilize a mono-oxyalkylene-functional silicone having at least one carbinol functional group to generally give an user a dry, silky, non-greasy lubricated feel when applied to the skin; that the application of such a lubricant can reduce the frictional qualities of the skin's surface as generally imparted by the moisture and oils present upon the skin's surface; and to generally ease the task of at least partially encapsulating a portion of the human body within flexible and elastic skin contact articles.

Although the description above contains many specifications, these should not be construed as limiting the scope of the invention but as merely providing illustrations of some of the presently preferred embodiments of this invention. Thus, the scope of the invention should be determined by the appended claims and their legal equivalents rather than by the examples given.

What is claimed is:

1. A method of lubricating the human skin comprising of the following steps, but not necessarily in the order shown:
(A) providing a composition of a gel formed from cross-linking siloxane polyethers, the gel being further micronized in one or more low viscosity, silicone fluids;
(B) applying an effective amount of the composition to a portion of the exterior surface of a skin; and
(C) reducing the frictional qualities of the portion.

2. The method of claim 1 wherein composition further contains one or more natural volatile oil astringents.

3. The method of claim 1 wherein the micronized gel's particle size may range from 0.2 to 50 microns.

4. The method of claim 1 further comprising of the step of contacting at least one flexible and elastic skin contact article to the portion.

5. The method of claim 4 further comprising of the step of moving the flexible and elastic skin contact article along the portion.

6. The method of claim 5 wherein moving the article along the portion results in at least a partial encapsulation of a hand within a glove.

7. The method of claim 5 wherein contacting the article along the portion results in at least a partial encapsulation of a limb within a compression wear.

8. The method of claim 1 wherein the cross-linked siloxane polyether is formed using a low viscosity organopolysiloxane selected from a set of organopolysiloxanes consisting of a low viscosity, volatile linear organopolysiloxane having from 2 to about 20 silicon atoms; low viscosity, volatile branched organopolysiloxane having from 2 to about 20 silicon atoms; and a cyclic organopolysiloxane having from 3 to about 6 silicon atoms.

9. The method of claim 1 wherein the siloxane polyethers are cross-linked by combining a low viscosity organopolysiloxane, an unsaturated organopolysiloxane, and a Si—H functional cross-linker.

10. The method of claim 9 wherein the low viscosity organopolysiloxane is present in an amount of 35% to 90% by weight; the unsaturated organopolysiloxane is present in an amount of about 5% to about 75% by weight, and the Si—H functional cross-linker is present in an amount of about 1-10% by weight.

11. The method of claim 9 wherein the low viscosity organopolysiloxane is present in an amount of 10% to 80% by weight; the unsaturated organopolysiloxane is present in an amount of about 10% to about 90% by weight, and the Si—H functional cross-linker is present in an amount of about 1-10% by weight.

12. The method of claim 1 wherein the low viscosity, silicone fluid is dimethylsiloxane.

13. A method using a skin lubricating composition comprising of the following steps, but not necessarily in the order shown:
(A) providing a skin lubrication composition comprising of at least one silicone gel, the silicone gel being selected from a group of silicone gels consisting of:
(i) a silicone gel formed from a silicone and a hydrosilylation compatible solvent wherein said silicone is prepared by the hydrosilylation of a linear alkenyl polyorganosiloxane and a hydride resin;
(ii) a silicone gel formed as a reaction product of an epoxy functional hydrido-siloxane said reaction product being formed in an epoxy-gel formation compatible solvent;
(iii) a silicone gel formed from a silicone and a hydrosilylation compatible solvent wherein said silicone is prepared by the hydrosilylation of a linear hydrogen organopolysiloxane having two or more hydride functionalities per molecule and an .alpha., .omega. reactive organic molecule possessing two or more reactive functionalities per molecule; and
(iv) a silicone gel formed as a reaction product of a vinyl functional hydrido-siloxane in a hydrosilylation compatible solvent.
(B) applying an effective amount of the composition to a portion of the exterior surface of a skin; and
(C) contacting the portion with at least a part of flexible and elastic skin contact article.

14. The method of claim 13 further comprising of the step of moving the part of the flexible and elastic skin contact article along the portion.

15. The method of claim 14 wherein moving the part of the elastic skin contact article results in at least a partial encapsulation of a human hand within a glove.

16. A method of claim 13 wherein the composition further contains micronized and dispersed particles of a hydrophilic polymer sized between one and one hundred microns that are capable of absorbing skin moisture.

17. A method of claim 16 wherein hydrophilic polymer particles upon contact with skin moisture within the vicinity of a perspiring human skin pore, rapidly gels and increases its size so as to create a temporary plug over and about or within said perspiring skin pore.

18. A method of lubricating the human skin comprising of the following steps, but not necessarily in the order shown:
(A) providing a mono-oxyalkylene-functional silicone having at least one carbinol functional group;
(B) applying an effective amount of the silicone to a portion of the exterior surface of the human skin; and
(C) contacting the portion with an elastic skin contact article to encapsulate at least a part of a human body.

19. The method of claim 19 further comprising of the step of moving the elastic skin contact article along the portion.

20. The method of claim 18 wherein moving the part of the elastic skin contact article results in at least partial encapsulation of a human hand within a glove.
